(19) United States
(12) Patent Application Publication
(10) Pub. No.: US 2010/0247529 A1
(43) Pub. Date: Sep. 30, 2010

(54) COOPERATIVE AND DYNAMIC ASSEMBLY OF AFFINITY COMPLEXES

(75) Inventor: William Patterson, Stoughton, WI (US)

Correspondence Address: Casimir Jones, S.C., 2275 DEMING WAY, SUITE 310, MIDDLETON, WI, 53562, US

(73) Assignee: William Patterson, Stoughton, WI (US)

(21) Appl. No.: 12/727,814

(22) Filed: Mar. 19, 2010

Related U.S. Application Data
(60) Provisional application No. 61/162,141, filed on Mar. 20, 2009.

(51) Int. Cl. A61K 39/395; C07K 2/00; C07K 14/00; C07K 16/00; C07K 16/46; C12N 9/00; A61P 43/00

(52) U.S. Cl. 424/133.1; 530/300; 530/350; 530/387.1; 530/387.3; 435/183

(57) ABSTRACT

The invention generally relates to the field of immunochemistry including antibody therapy, diagnostics, and basic research and specifically relates to the area of alternatives to natural antibodies including artificial antibodies or antibody mimics. The invention relates particularly to the cooperative assembly of stable affinity complexes.

This application claims priority to U.S. Provisional Patent Application Ser. No. 61/162,141, filed Mar. 20, 2009, which is herein incorporated by reference in its entirety.

### FIELD OF THE INVENTION

The invention generally relates to the field of immunochemistry including antibody therapy, diagnostics, and basic research and specifically relates to the area of alternatives to natural antibodies including artificial antibodies or antibody mimics. The invention relates particularly to the cooperative assembly of stable affinity complexes.

### BACKGROUND OF THE INVENTION

Monoclonal antibodies have been important tools in immunochemistry for more than three decades. The discovery of monoclonal antibodies more than 30 years ago provided the foundation for a fast emerging sector of biopharmaceuticals. Although the first murine monoclonal antibody (Orthoclone OKT3) was approved in 1986, it was the development of mouse/human chimera and the humanization of mouse monoclonal antibodies that led to the acceptance of these therapeutic alternatives 10 years later. There are now 21 antibodies approved by the FDA, and close to 30 globally, with more than 200 more in clinical trials and nearly half of those in phase II and III. Global sales of antibody therapeutics in 2008 were over $30 billion with 8 drugs surpassing $1 billion blockbuster status. Although the main pharmaceutical markets for this therapy has been and continues to be oncology and AIID (autoimmune and infectious disease), expansion into rheumatology, allergy, cardiology, and transplantation markets are expected. Therapeutic antibodies are typically considered less toxic than small molecules because of their homology to endogenous human IgG and their designed specificities. Products can be developed rapidly, conjugated to expand their effectiveness (chemo- and radiotherapy), and have a high rate of success in the clinical trial process. However, recombinant antibody molecules are relatively large (150 kDa), complex (4 domains) molecules that require specific disulfide bond formation and site-specific glycosylation. Therapeutic antibodies are normally expressed in established mammalian systems, such as CHO (Chinese hamster ovary) cells, in relatively modest amounts requiring very large volumes and strict conditions. Product purification and characterization can be equally demanding as all traces of media and cell components must be removed and the homogeneity of the glycoproteins verified. These characteristics make antibodies challenging and expensive to manufacture, which has contributed to the exploration for alternatives. Scaffolds for antibody mimics are typically much smaller proteins (3-20 kDa), many do not require disulfide bond formation or glycosylation, and can be expressed in highly productive bacterial systems.

While the natural IgG molecule is the standard tool for both diagnostic and therapeutic applications, alternatives have been developed to improve and expand their functionality. Examples of these early advances include Fab fragments, single chain variable fragments (scFv), and V_{H}H domains (e.g., Nanobodies) from dromedaries. The affinity of these binders, which are 15 to 30% the size of an IgG, can be improved by in vitro techniques, taking advantage of advancements in gene library construction and various panning technologies, such as in vivo phage, yeast, and E. coli display or the in vitro ribosome, RNA, and DNA display technologies. These technologies have allowed the development of numerous protein scaffolds that have a unique affinity interaction domain that can bind with target epitopes. The list of scaffolds is extensive, but only a few have been developed commercially. For example, an Affibody is derived from the Z-domain of the S. aureus Protein A, the Adnectin (or Monobody) from the 10th fibronectin type III domain, the Anticalin from lipocalin, and a DARPin from the ankyrin repeat protein. These artificial antibodies can require large libraries and significant screening to identify a binder with effective affinity.

The average protein target can have numerous epitopes and a multi-component complex can have dozens of epitopes. Therefore, it is possible to generate a diverse array of artificial antibodies that can bind a target and these binders can be linked together to increase their affinity and specificity through avidity. For example, Avimers are a string of 3 small, single domains that recognize different and discontinuous epitopes of a single target (Silverman, J., et al., Nature Biotech. 23, 1556). Avimers are created by panning for a single binding domain from a phage display library under low stringency conditions, then employing exon shuffling to connect another binding domain from a similar library that is linked to the first domain via a linker. This is repeated once more to generate the 3 domains of the Avimer. While each domain has relatively weak or moderate affinity for its epitope, the combined affinities, or avidity, of the Avimer is very strong.

Other researchers have increased binding affinity through avidity by fusing self-assembling polypeptide sequences to small domains with moderate affinity that generate multivalent complexes similar to IgM antibody complexes. For example, homotrimeric molecules (e.g. tetranectin) contain three binding domains per molecule. Self-assembly molecules have been developed using the B-subunit of the E. coli verotoxin, which forms a homopentamer (US Pat. App. No. 2006/0051292). In one case, the increase in binding due to avidity was greater than 7000-fold (Zhang et al., J. Mol. Biol. 335:49-56). These self-assembling IgM-like molecules are limited by their single epitope interaction motif, that is, they are homomultimers.

Multicomponent macromolecular complexes abound in nature and are integral to biochemical function within cells, cellular systems, and organisms (Williamson, Nat. Chem. Biol. 4, 458-465). Many of these complexes are transient, dynamic, and cooperative, which requires that the components of the complex have relatively moderate affinity for each other. The laws of thermodynamics dictate the association and dissociation of small molecules, macromolecules, and molecular complexes. Creating artificial multicomponent complexes with configurational cooperativity can be very challenging due to molecular spacing and orientation, requiring a balance of molecular flexibility and stability

### SUMMARY

In some embodiments, the present invention provides an affinity complex composition comprising: (a) two or more primary affinity molecules, wherein the primary affinity molecules have affinity for one or more target molecules, and (b) one or more linker affinity molecules, wherein the linker affinity molecules have affinity for two or more of the primary affinity molecules, wherein the linker affinity molecules form a bridge when bound to two or more of said primary affinity molecules. In some embodiments, the affinity complex composition preferentially assembles into a complex upon binding to a target molecule. In some embodiments, the primary affinity molecules are one or more of natural antibodies, antibody fragments, single chain variable fragments, Nanobodies, Affibodies, Anticalins, DARPins, Monobodies, Avimers, and Microbodies. In some embodiments, the primary affinity molecules are expressed from genes. In some embodiments, the primary affinity molecules comprise the same type of affinity molecule. In some embodiments, the primary affinity molecules comprise two or more different types of affinity molecules. In some embodiments, the two or more primary affinity molecules bind to different epitopes on the target molecule. In some embodiments, the two or more primary affinity molecules bind to the same epitope on a target molecule. In some embodiments, the target is a homomultimer and the epitopes are discontinuous. In some embodiments, the linker affinity molecule comprises two or more secondary affinity domains that are linked. In some embodiments, the secondary affinity domains bind the primary affinity molecules at an epitope. In some embodiments, the binding of the secondary affinity domains to the primary affinity molecules does not disrupt the function of the primary affinity molecules. In some embodiments, the secondary affinity domains of the linker affinity molecule are covalently attached by a flexible linker. In some embodiments, the flexible linker is one or more of a polypeptide, a nucleic acid strand, polyethylene glycol, and peptide nucleic acid (PNA). In some embodiments, the secondary affinity domains and the flexible linker comprise a single polypeptide. In some embodiments, the secondary affinity domains and the flexible linker are attached through a non-covalent interaction. In some embodiments, the affinity complex composition further comprises one or more accessory molecules. In some embodiments, accessory molecules are one or more of fluorescent molecules, radioactive molecules, enzymes, inhibitors, drug molecules, and cell adhesion molecules. In some embodiments, the accessory molecules are bound to the linker affinity molecule. In some embodiments, the accessory molecules are bound to one or more of said primary affinity molecule. In some embodiments, the accessory molecules comprise two or more fluorescent molecules linked to two or more affinity molecules. In some embodiments, FRET can be detected between the fluorescent molecules upon formation of the affinity complex composition. In some embodiments, the accessory molecules comprise a drug molecule, wherein said drug molecule is incorporated into a cell upon interaction of the affinity complex composition with the target. In some embodiments, the linker affinity molecule comprises an affinity complex comprising or consisting of two or more secondary affinity molecules, wherein the secondary affinity molecules have affinity for each other. In some embodiments, the secondary affinity molecules comprise coiled coil peptide sequences that interact in a specific manner. In some embodiments, the secondary affinity molecules comprise complementary nucleic acid sequences. In some embodiments, the secondary affinity molecule has affinity for a tertiary affinity molecule. In some embodiments, binding of the tertiary affinity molecule to the secondary affinity molecules does not interfere with binding of the secondary affinity molecules to the primary affinity molecules. In some embodiments, the secondary affinity molecules interact with more than one domain of the primary affinity molecule. In some embodiments, the affinity complex composition comprises a tyrosine/serine binary-code interface. In some embodiments, the affinity complex composition comprises a tyrosine/serine/X amino acid tertiary-code interface. In some embodiments, the interaction of the paratope of the primary affinity molecule to the epitope of the target molecule causes a change in the conformation of the primary affinity molecule to increase the affinity of the linker interaction domain with the linker affinity molecule. In some embodiments, standard cellular process causes a change in the conformation of the primary affinity molecule to increase the affinity of its linker interaction domain with the linker affinity molecule. In some embodiments, standard cellular process comprises a protease, kinase, or phosphatase reaction. In some embodiments, standard cellular process comprises a metal binding event. In some embodiments, the metal comprises zinc, calcium, magnesium, iron, or cobalt. In some embodiments, the affinity complex composition comprises three primary affinity molecules, wherein each of the primary affinity molecules bind to discontinuous epitopes of the target, and wherein the linker affinity molecules bind to said primary affinity molecules through secondary affinity molecules.

The target or target molecules may comprise a single protein or other biomolecule or multiple molecules (e.g., in a multi-molecular complex). For example, in some embodiments, affinity molecules are used to simultaneously bind two or more molecules that are in proximity to one other, to, for example, detect such proximity.

Embodiments of the present invention further provide methods of using the complexes in therapeutic, diagnostic, and basic or applied research settings (e.g., drug screening applications).

### BRIEF DESCRIPTION OF THE DRAWINGS

The foregoing summary and detailed description is better understood when read in conjunction with the accompanying drawings which are included by way of example and not by way of limitation.

FIG. 1 shows a cartoon representing a simple design of an affinity complex comprising two primary affinity molecules, a target, and a linker affinity molecule.

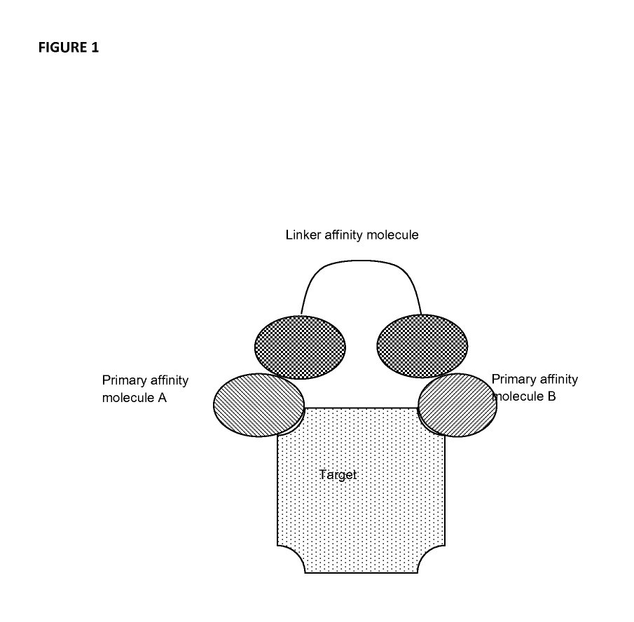

FIG. 2 shows the thermodynamic cycle of a quaternary affinity complex where “T” is the target molecule, “A” and “B” are the primary affinity molecules, “L-L” is the linker molecule, “kₐ” is the association constant, and “k_{d}” is the dissociation constant. The change in free energy for each interaction is represented by the formula ΔG°=−RTln K where K=k_{d}/kₐ.

### DEFINITIONS

As used herein, the term “about” means encompassing plus or minus 10%. For example, about 200 nucleotides refers to a range encompassing between 180 and 220 nucleotides.

As used herein, the term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment is retained. As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to nucleic acid sequences that are not found naturally associated with the gene sequences in the chromosome or are associated with portions of the chromosome not found in nature (e.g., genes expressed in loci where the gene is not normally expressed).

As used herein, the term “nucleic acid molecule” refers to any nucleic acid containing molecule, including but not limited to, DNA or RNA. The term encompasses sequences that include any of the known base analogs of DNA and RNA including, but not limited to, 4 acetylcytosine, 8-hydroxy-N-6-methyladenosine, aziridinylcytosine, pseudoisocytosine, 5 (carboxyhydroxyl-methyl) uracil, 5-fluorouracil, 5 bromouracil, 5-carboxymethylaminomethyl 2 thiouracil, 5 carboxymethyl-aminomethyluracil, dihydrouracil, inosine, N6 isopentenyladenine, 1 methyladenine, 1-methylpseudo-uracil, 1 methylguanine, 1 methylinosine, 2,2-dimethyl-guanine, 2 methyladenine, 2 methylguanine, 3-methyl-cytosine, 5 methylcytosine, N6 methyladenine, 7 methylguanine, 5 methylaminomethyluracil, 5-methoxy-amino-methyl 2 thiouracil, beta D mannosylqueosine, 5′ methoxycarbonylmethyluracil, 5 methoxyuracil, 2 methylthio N6 isopentenyladenine, uracil 5 oxyacetic acid methylester, uracil 5 oxyacetic acid, oxybutoxosine, pseudouracil, queosine, 2 thiocytosine, 5-methyl-2 thiouracil, 2-thiouracil, 4 thiouracil, 5-methyluracil, N-uracil 5 oxyacetic acid methylester, uracil 5 oxyacetic acid, pseudouracil, queosine, 2-thiocytosine, and 2,6 diaminopurine.

As used herein, the term “oligonucleotide” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. To further illustrate, oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Typically, the nucleoside monomers are linked by phosphodiester bonds or analogs thereof, including phosphorothioate, phosphorodithioate, phosphoroselenoate, phosphorodiselenoate, phosphoroanilothioate, phosphoranilidate, phosphoramidate, and the like, including associated counterions, e.g., H⁺, NH₄⁺, Na⁺, and the like, if such counterions are present. Further, oligonucleotides are typically single-stranded. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (1979) Meth Enzymol. 68:90-99; the phosphodiester method of Brown et al. (1979) Meth Enzymol. 68:109-151; the diethylphosphoramidite method of Beaucage et al. (1981) Tetrahedron Lett. 22:1859-1862; the triester method of Matteucci et al. (1981) J Am Chem. Soc. 103:3185-3191; automated synthesis methods; or the solid support method of U.S. Pat. No. 4,458,066, entitled “PROCESS FOR PREPARING POLYNUCLEOTIDES,” issued Jul. 3, 1984 to Caruthers et al., or other methods known to those skilled in the art. All of these references are incorporated by reference.

As used herein, the term “sample” refers to anything capable of being analyzed by the methods provided herein. In some embodiments, the sample comprises or is suspected one or more nucleic acids capable of analysis by the methods. Preferably, the samples comprise nucleic acids (e.g., DNA, RNA, cDNAs, etc.) from one or more Salmonella enterica strains or isolates. Samples can include, for example, evidence from a crime scene, blood, blood stains, semen, semen stains, bone, teeth, hair saliva, urine, feces, fingernails, muscle tissue, environmental samples, water samples, cigarettes, stamps, envelopes, dandruff, fingerprints, personal items, swab from a NICU, swab from a ventilator, sputum, wound samples, respiratory samples, cultures of samples and the like. In some embodiments, the samples are “mixture” samples, which comprise nucleic acids from more than one subject or individual. In some embodiments, the methods provided herein comprise purifying the sample or purifying the nucleic acid(s) from the sample. In some embodiments, the sample is purified nucleic acid.

A “sequence” of a biopolymer refers to the order and identity of monomer units (e.g., nucleotides, etc.) in the biopolymer. The sequence (e.g., base sequence) of a nucleic acid is typically read in the 5′ to 3′ direction.

As used herein, the term “affinity complex” refers to an interacting multicomponent collection of molecules that specifically interacts through interactions (e.g. hydrogen bonding, Van der Waals forces, electrostatic forces, hydrophobic forces, etc.) with a target molecule.

As used herein, the term “affinity molecule” refers to any molecule that specifically interacts through interactions (e.g. hydrogen bonding, Van der Waals forces, electrostatic forces, hydrophobic forces, etc.) with a target molecule.

As used herein, the term “artificial antibody” or “antibody mimic” refers to any non-immunoglobulin molecule or molecular complex that is created to specifically interact with a target molecule.

As used herein, the term “epitope” refers to any surface region of a target molecule to which an affinity molecule binds.

As used herein, the term “discontinuous epitopes” refers to two or more surface regions of a target molecule or molecules that are separated by a defined distance.

The term “paratope” refers to the surface region of an affinity molecule that interacts with the epitope of the target molecule.

As used herein, the term “affinity” refers to the non-random interaction of two molecules. The term “affinity” refers to the strength of interactions and can be expressed quantitatively as a dissociation constant (K_{D}). One or both of the two molecules may be a peptide (e.g. antibody). Binding affinity (i.e., K_{D}) can be determined using standard techniques. For example, the affinity can be a measure of the strength of the binding of an individual epitope with an antibody molecule.

As used herein, the term “avidity” refers to the cooperative and synergistic bonding of two or more molecules. “Avidity” refers to the overall stability of the complex between two or more populations of molecules, that is, the functional combining strength of an interaction.

### DETAILED DESCRIPTION OF EMBODIMENTS

In some embodiments, the present invention provides compositions, systems, and methods related to affinity complexes. In some embodiments, the present invention provides compositions, systems, and methods related to affinity complexes configured to form in a cooperative and dynamic process in the presence of a target. In some embodiments, a target contains two or more discontinuous epitopes to which the affinity complex interacts. In some embodiments, the affinity complex comprises or consists of three or more components: (1) a primary affinity molecule that interacts with one epitope of the target, (2) a second primary affinity molecule that interacts with a second epitope of the same target, and (3) a linker that interacts with the 2 primary affinity molecules.

In some embodiments, two or more primary affinity molecules bind to epitopes on a target molecule. In some embodiments, the epitopes are discontinuous. In some embodiments, the primary affinity molecules recognize the same epitopes. In some embodiments, the primary affinity molecules recognize different epitopes. In some embodiments, a linker affinity molecule comprises two or more secondary affinity domains. In some embodiments, a linker affinity molecule comprises two or more secondary affinity domains connected by one or more flexible linkers. In some embodiments, each secondary affinity domain of the linker affinity molecule recognizes and binds to a primary affinity molecule of the present invention. In some embodiments, secondary affinity domains on the linker affinity molecule recognize the linker interaction domain of the primary affinity molecules. In some embodiments, two or more primary affinity molecules of the invention have the same linker interaction domains. In some embodiments, two or more primary affinity molecules of the invention have different linker interaction domains. In some embodiments, linker affinity molecules of the present invention bind to two or more primary affinity molecules through interactions between secondary affinity domains and linker interaction domains. In some embodiments, the cooperativity of the complex of interactions of the present invention increases the specificity of target recognition and binding. In some embodiments, the cooperativity of the complex of interactions of the present invention increases the affinity and strength of target recognition and binding.

In some embodiments, the present invention comprises a more complex set of interactions, as described below. In some embodiments, an affinity complex of the present invention provides recognition of multiple targets. In some embodiments, an affinity complex of the present invention provides multiple different linker affinity molecules. In some embodiments, an affinity complex of the present invention provides linker affinity molecules that recognize and bind multiple different primary affinity molecules. In some embodiments, an affinity complex of the present invention provides linker affinity molecules which recognize and bind multiple different linker interaction domains.

### Primary Affinity Molecules

In some embodiments, primary affinity molecule comprises or consists of a scaffold that has a target epitope interaction domain or region known as a paratope, and a linker interaction domain or region. In some embodiments, the paratope and linker interaction domain are situated to allow interaction with a target epitope and a linker molecule. In some embodiments, each primary affinity molecule can comprise or consist of the same scaffold. In some embodiments, each primary affinity molecule can comprise or consist of different scaffolds.

In some embodiments, primary affinity molecules can be any antibody, antibody fragment, scaffold or molecular construct that has a paratope domain or region and a linker interaction domain or region. For example, IgG antibodies known to interact with a single target at two discontinuous epitopes can used with a linker molecule that interacts with each Fc domain of the IgG (such as Protein A or G). The thermodynamic kinetics for this complex should take into account that each IgG antibody contains 2 binding domains. In some embodiments, Fab fragments of an IgG antibody are employed as the primary affinity molecule. In embodiments where, Fab fragments of an IgG antibody are employed as the primary affinity molecule, the preferred linker molecule would interact preferentially with the constant domains (CL and CH1) of the molecule. In some embodiments, single chain fragments of the variable domains (scFv) are employed due to their increased stability. In some embodiments, the smaller size of the VHH domain of camelids (Nanobodies) is a preferred affinity molecule.

In some embodiments, the primary affinity molecule is a Monobody (fibronectin type III domain) derived from a human cell surface protein. This scaffold is structurally similar to antibody variable domains, but does not contain disulfide bonds that can hinder expression in prokaryotic systems. In some embodiments, monobodies have a molecular weight of ˜10,000 Daltons, they are very soluble, and thermally and proteolytically stable. In some embodiments, the monobody scaffold contains three loops (BC, DE, and FG loops) that can be collectively employed as a paratope, similar to the CDR regions of an immunoglobulin. The polar opposite end of the paratope region contains three additional loops (AB, CD, and EF loops). In some embodiments, the AB, CD, and EF loops can be employed as interaction domains of the secondary affinity reagent. In some embodiments, monobodies are the secondary affinity molecules of the present invention. In some embodiments, monobodies serving as secondary affinity molecules can be linked as described above, or expressed as a single polypeptide with the C-terminus of one monobody linked to the N-terminus of the other monobody using a glycine/serine linker. In some embodiments, other linkers can be used, such as an abbreviated rPEG.

In some embodiments, the primary affinity molecule is a DARPin (designed ankyrin repeat protein) that is derived from a large class of repeat proteins found in various cellular sections in a variety of species. Each repeat consists of 33 amino acid residues that form a beta-turn followed by two anti-parallel helices and a randomized loop that is joined to the beta-turn of the next repeat and functions to “stack” the repeats generating a very stable hydrophobic core. In some embodiments, the loop and beta-turn sequences are involved in the paratope of the molecule. In some embodiments, residues of the helices can contribute to the paratope. In some embodiments, the combination of the loop and beta-turn sequences and the residues of the helicies generate a broad paratope interface. In some embodiments, three or more of these repeats are created to generate a molecule with very high affinity. In some embodiments, the ends of the repeats are “capped” to preserve the hydrophobic core, increase its solubility and stability, and can be used for labeling or immobilization. In some embodiments, N-terminal and C-terminal caps are employed as interaction domains of the secondary affinity reagent.

In some embodiments, the primary affinity molecule is an Affibody (the Z domain of Staphylococcal protein A) that comprises or consists of 58 amino acids arranged as a bundle of 3 anti-parallel alpha helices. In some embodiments, the small size of the affibody molecule provides easier expression and solubility in prokaryotic systems. In some embodiments, the affibody polypeptide is chemically synthesized then folded, which allows the introduction of non-canonical amino acids in the interaction domain or the addition of labels or reactive groups. In some embodiments, the interaction domain comprises or consists of 13 amino acid residues that are randomized to generate a library from which an affinity molecule is panned. In some embodiments, binding affinities for affibodies and their substrates are in the nanomolar range.

In some embodiments, the primary affinity molecule is a Microbody (Nascacell Technologies). In some embodiments, a microbody is based on natural cysteine-knot microproteins and cyclical knottins. In some embodiments, Microbodies are small (28 to 45 amino acids), yet very stable due to three disulfide bonds within the structure, which allows the display of a single peptide loop up to 20 amino acids. In some embodiments, microbodies are very soluble and are expressed from bacteria or synthesized by chemical means then properly folded. In some embodiments, the stability and solubility of these proteins provides alternative therapeutic delivery modes to the standard injection of most biologicals. In some embodiments, a very similar molecule called a Versabody (Amunix), acts as a primary affinity molecule. In some embodiments, a Versabody is a very small high disulfide density scaffold based on natural biopharmaceuticals, such as scorpion toxin. Versabodies are extremely stable, soluble, and non-immunogenic.

In some embodiments, the primary affinity molecule is an Anticalin, an Avimer or the domain A of an Avimer, a thioredoxin, an ubiquitin, a gamma-crystallin, CTLA-4 (Evibody), or other recombinant artificial antibodies. In some embodiments, a primary affinity molecule is any molecule capable of binding a target with a suitable affinity.

In some embodiments, primary affinity molecules have an auxiliary interaction domain or region added to their structure that allows the binding of an auxiliary functional group (e.g. detectable label, radioisotope, drug, toxin, enzyme or other moiety). In some embodiments, for example, the C-terminus of a fibronectin type III scaffold is extended with the Nano-tag peptide sequence that binds streptavidin with nanomolar affinity and labeled streptavidin conjugates added to bind the affinity molecules. In some embodiments, an auxiliary interaction domain is added to a linker molecule of the present invention.

### Linker Affinity Molecules

In some embodiments, the linker molecule comprises or consists of two or more (e.g. 2, 3, 4, 5, 6, 7, 8, 9, 10, 11, 12, 13, 14, 15, 16, 17, 18, 19, 20, . . . , 30, . . . , etc.) linked secondary affinity molecules that interact with the interaction domains of a primary affinity molecule. In some embodiments, the configurational cooperativity or avidity of the complex assembly is dependent on the affinity of the secondary affinity molecules for the primary affinity molecules. In some embodiments, the configurational cooperativity or avidity of the complex assembly is dependent on the degree of freedom of each affinity molecule. In some embodiments, the secondary affinity molecules of the linker molecule are covalently linked via a flexible polymer such as a polypeptide (e.g. glycine/serine polypeptides), a nucleic acid strand, polyethylene glycol, and peptide nucleic acid (PNA) that has sufficient degree of freedom to allow the interaction of the secondary affinity molecules with the primary affinity molecules. In some embodiments, the secondary affinity molecules of the linker molecule are linked, either directly or linked via a suitable linker. The present invention is not limited to any particular linker group. Indeed, a variety of linker groups are contemplated, suitable linkers could comprise, but are not limited to, alkyl groups, ether, polyether, alkyl amide linker, a peptide linker, a modified peptide linker, a Poly(ethylene glycol) (PEG) linker, a streptavidin-biotin or avidin-biotin linker, polyaminoacids (eg. polylysine), functionalised PEG, polysaccharides, glycosaminoglycans, dendritic polymers such as described in WO93/06868 and by Tomalia et al. in Angew. Chem. Int. Ed. Engl. 29:138-175 (1990), PEG-chelant polymers such as described in W94/08629, WO94/09056 and WO96/26754, oligonucleotide linker, phospholipid derivatives, alkenyl chains, alkynyl chains, disulfide, or a combination thereof.

In some embodiments the linker comprises a single chain connecting secondary affinity molecule to a second secondary affinity molecule. In some embodiments, there are multiple linkers connecting secondary affinity molecules to a single secondary affinity molecule. In some embodiments, a linker may connect multiple secondary affinity molecules to each other. In some embodiments, a linker attaches an additional functional portion to secondary affinity molecule. In some embodiments, a linker may be branched, connecting more than two secondary affinity molecules. In some embodiments, the linker may be flexible, or rigid. In some embodiments, the linker of the present invention is cleavable or selectively cleavable. In some embodiments, the linker is cleavable under at least one set of conditions, while not being substantially cleaved (e.g. approximately 50%, 60%, 70%, 80%, 90%, 95%, 99%, or greater remains uncleaved) under another set (or other sets) of conditions. In some embodiments, the linker is susceptible to enzymatic cleavage (e.g. proteolysis). In some embodiments, the enzymatic cleavage is site specific (e.g. sequence specific). In some embodiments, the enzymatic cleavage is at a random site along the linker. In some embodiments, the enzymatic cleavage may occur at multiple random sites along the linker. In some embodiments, the linker is susceptible to cleavage under specific conditions relating to pH, temperature, oxidation, reduction, UV exposure, exposure to radical oxygen species, chemical exposure, light exposure (e.g. photo-cleavable), etc.

In some embodiments, an affinity complex of the present invention provides high avidity binding and greater specificity than do the interactions of the two discontinuous target epitopes. As a result, primary affinity molecules with moderate affinities (>10 nM) are preferred.

In some embodiments, linker molecules that can form a complex with primary affinity molecules are be prepared by any suitable method. In some embodiments, IgG antibodies are used as affinity molecules, and anti-Fc antibodies or Fab fragments can be linked via their reduced thiol groups, preferably using a crosslinking system from SoluLink. In some embodiments, one part of the anti-Fc IgG/Fab is labeled with MHPH (3-N-Maleimido-6-hydraziniumpyridine hydrochloride) and the other part with MTFB (Maleimido trioxa-6-formyl benzamide). In some embodiments, the hydrazine moiety of the MHPH-modified molecules react with 4-formylbenzamide of the MTFB-modified molecules to form stable bis-arylhydrazone-mediated conjugates. In some embodiments, alternative methods for crosslinking proteins, known to those skilled in the art, are utilized. In some embodiments, an oligonucleotide can be synthesized with chemically reactive moieties (for example, a maleimide) on each end that would react with the secondary affinity molecule. In some embodiments, each molecule could be conjugated to an oligonucleotide, one with a 3′ is free and the other with a 5′ free so that the two strands can be ligated. In some embodiments, any suitable methods to link the secondary affinity molecules would also be appropriate so long as the linker has flexibility to allow interaction of the secondary affinity molecules.

In some embodiments, an affinity complex comprises a quaternary system with four components: a target, two primary affinity molecules and a secondary affinity molecule. In some embodiments, more intricate affinity complex designs, involving more than 4 components are used (e.g. 5 components, 6 components, 7 components, 8 components, 9 components, 10 components, 11 components, 12 components, 13 components, 14 components, 15 components, 16 components, 17 components, 18 components, 19 components, 20 components, >20 components, etc.). In some embodiments, a quinary system (i.e. 5 components) could consist of a target, 2 primary affinity molecules, and 2 secondary affinity molecules (linker affinity molecules) that have domains that can freely interact. An additional affinity molecule that labels the complex, binding to either primary affinity molecule or the linker with its secondary affinity molecules, provides another example of a quinary system. In some embodiments, 3 primary affinity molecules that interact with 3 discontinuous epitopes of the target and are assembled with a secondary linker that interacts with each primary affinity molecule. The complexity and component possibilities are significantly amplified with senary (6 components), septenary (7 components), octonary (8 components), nonary (9 components), and denary 10 components) systems.

In some embodiments, a linker complex in a quinary system is heterodimeric helical coiled-coil domains, where one of the affinity molecules has a K-coil, preferable to the N- or C-terminus, while the other affinity molecule has an E-coil attached or fused. The multimer heptad K-coil naturally forms an alpha helix and aligns the positive charges of the lysine (K) amino acids on one side of the coil. The multimer heptad E-coil does the same, except it aligns the negative charges of the glutamic acid (E) amino acids. The positive charges of the K-coil form salt bridges with the negative charges of the E-coil and the binding interaction strength is determined by the number of heptads (number of salt bridges).

In some embodiments, a linker complex is a single stranded DNA covalently attached to each secondary affinity molecule. In some embodiments, the free end of the DNA strand of one affinity molecule would be complementary to the free end of the other affinity molecule. In some embodiments, hybridization of the complementary DNA strands would provide formation of the linker. In some embodiments, Protein:oligonucleotide conjugate linkers can be prepared by many different methods (e.g. by labeling the protein with a moiety that reacts with a moiety placed on the end of the synthetic oligonucleotide).

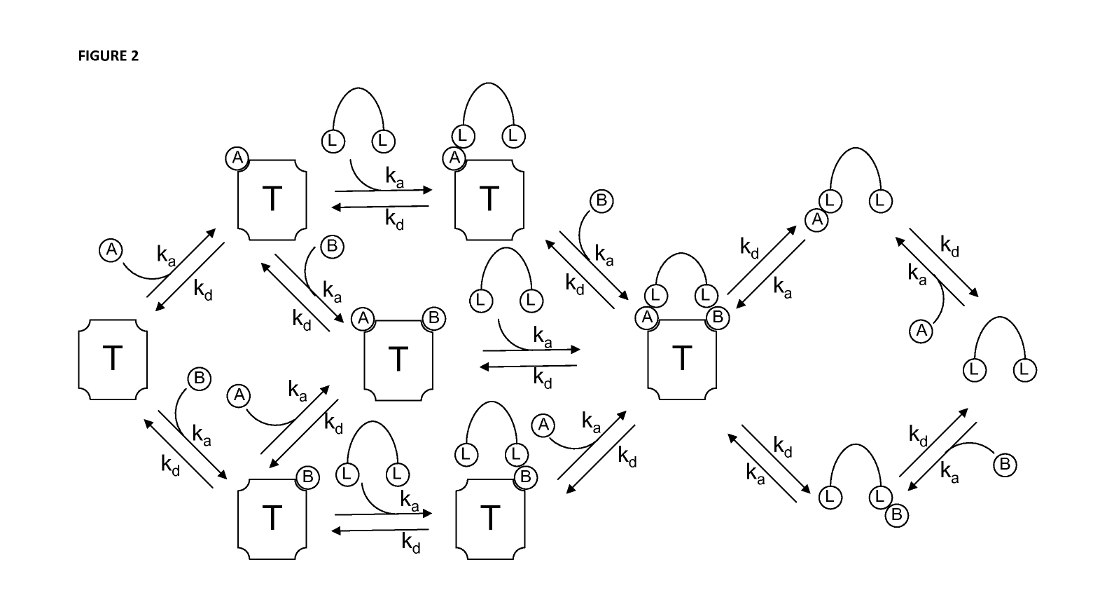

In some embodiments, the thermodynamics of the affinity complex assembly are an important aspect of the present invention. FIG. 2 represents a thermodynamic cycle of a quaternary system where the primary affinity molecules interact with the epitopes of a target and also with the linker. Association and dissociation constants of each interaction are represented by kₐ and k_{d}, respectively, and together determine the affinity (K_{D}) of the interaction. The free energy of formation is symbolized by ΔG° and is equal to −RTlnK_{D}. Each interaction contributes to the formation of the complex in a cooperative manner, which results in an interaction called avidity that corresponds to coupling of the free energies, or ΔΔG. In some embodiments, the affinity constants within a complex are also dependent on molecular “stress” of the system, including the intramolecular flexibility of the components, intermolecular alignment within the complex, and steric hindrance in binding. All of these parameters contribute to the ΔΔG.

### Paratopes of Cooperative Affinity Complexes

In some embodiments, the binding interaction of a paratope to its epitope is based upon a combination of molecular contacts that together account for the affinity strength (e.g. Van der Waals interactions, hydrogen bonding, and hydrophobic interactions), specific amino acid side groups of the paratope polypeptide form bonds with amino acid side groups of the epitope polypeptide. In some embodiments, a portion of the amino acids in the paratope function as structural support. In some embodiments, antibody mimics have a single polypeptide paratope, such as Affibodies and Versabodies. In these embodiments, the sum of those interactions determines the affinity. In some embodiments, affinity molecules comprise multiple polypeptide loops or CDRs (complementarity determining regions), such as fibronectin Type III domains, ankyrin repeats, and IgG molecules. These embodiments demonstrate additional number and spacing of those interactions. In some embodiments, the structure of the paratope should be adaptable to fit the epitope. In some embodiments, the paratope has enough flexibility to form bonds with the epitope without introducing intramolecular strain. In some embodiments, a large number of affinity molecules are be screened (e.g., in a binding assay) to achieve a suitable structure.

In some embodiments, preparing and panning affinity molecules from translated DNA libraries to identify those with high affinity and specificity carried out as part of the creation of an affinity mimic. There are numerous methods for performing this function. In some embodiments, preparing and panning the affinity mimic is linked to its genetic code in by some means. Some embodiments use in vivo display techniques, such as phage, yeast, and bacterial display, where the affinity molecule is “displayed” on the surface of a cell or virus based on the genetic code inside. In some embodiments, in vitro display methods that use translation cocktails, such as S30, wheat germ, or rabbit reticulocyte lysates, to express the affinity polypeptide, while retaining its association to the nucleic acid, are used. In some embodiments, ribosome display is used. In ribosome display, the ribosome structure is the connection between the mRNA and polypeptide while with RNA display it is a linker attached to the 3′ end of the mRNA that attaches to the polypeptide. In some embodiments DNA display is used. For DNA display, a polypeptide is co-expressed with the affinity molecule that specifically binds to the DNA coding region that transcribed its mRNA. In some embodiments, one of several in vivo techniques that express both the antigen (target) and the affinity binder within the cell and the binding events are detected by activation of a reporter gene (two-hybrid techniques) or by protein-fragment complementation assay. In some embodiments, the size and diversity of the coding library that expresses the affinity mimics are a key to the method used. In some embodiments, numerous rounds of affinity molecule “enhancement” using sophisticated gene sequence shuffling is required to achieve high affinity binding interactions.

In some embodiments, only moderate affinity interactions are required. In some embodiments, only moderate affinity interactions are preferred. In embodiments, increased effectiveness of screening libraries is achieved when moderate affinity is sought. In some embodiments, binary- or tertiary-code library systems reduce the size of the libraries, increase their effectiveness, and further simplify the process. In some embodiments, the basis of the binary-code interface within affinity molecules is that effective affinity binders can be generated by using only 2 amino acids, tyrosine and serine (e.g. fibronectin type III domains that were developed using the Tyr/Ser binary-code interface demonstrated affinities to 3 different proteins of 5 to 90 nM (Koide, A., et al., Proc. Nat. Acad. Sci. 104, 6632-6637, herein incorporated by reference in its entirety)). In some embodiments, a nanomolar affinity level, which can be achieved in binary-code interface, is very effective in an affinity complex where the binding affinities are multiplied by the linkage of the affinity molecules. In some embodiments, the combination of a simplified binary-code interface library system and a cooperative affinity complex system greatly reduces the time and resources necessary to development high affinity and specific affinity complexes.

### Cooperative Assembly of Complexes

In some embodiments, binding interactions are observed by labeling the components (e.g. with small molecule fluorescent probes). In some embodiments, fluorescent molecules with distinct fluorescence characteristics (excitation and emission) and reactive moieties for covalent linkage to proteins can be utilized to label proteins (e.g. Alexa Fluors (Invitrogen), CyDye Fluors (GE Healthcare Life Sciences), DyLight Fluors (Dyomics GmbH), HiLyte Fluors (Anaspec) and the IRDye Near Infrared Fluors (Li-Cor)). In some embodiments, fluorophores are linked to either a NHS ester reactive group (reacts with ε-amine of lysine and the α-amine of the polypeptide N-terminal) or a maleimide reactive group (reacts with reduced sulfhydryl of cysteine). In some embodiments, labeling proteins non-specifically, especially small polypeptides can potentially interfere with their function. In some embodiments, it is important to demonstrate no loss of utility of the affinity molecule. In some embodiments, if the affinity molecule does not have a cysteine in the polypeptide sequence (such as an Affibody or fibronectin scaffold), a cysteine can be introduced at the C-terminal and specifically labeled with any maleimide fluorophore.

In some embodiments, the affinity molecules and linker are each covalently labeled with a distinct fluorescent molecule, each with different fluorescent properties, so that each could be detected simultaneously, to determine binding of each component. In some embodiments, the target is immobilized on a surface (e.g. plastic, glass, nitrocellulose, etc.), the surface blocked to reduce non-specific binding, and the components of the affinity complex allowed to bind. In some embodiments, the binding surface is scanned by a fluorometer (plate or slide reader, or digital camera imager) to detect the fluorescence. In some embodiments, a fluorescent bead format, where the target is immobilized onto the surface of a bar-coded bead and the binding events detected by flow cytometry, is utilized.

In some embodiments, the formation of the affinity complex is detected using FRET (fluorescence resonance energy transfer) or BRET (bioluminescence resonance energy transfer). FRET and BRET technologies are homogeneous assays, in which the target does not need to be immobilized. In some embodiments, when using FRET, one of the primary affinity molecules is labeled with a donor fluorophore and either the other primary affinity molecule or the linker is labeled with an acceptor fluorophore. The fluorophores could be either fluorescent dyes or proteins. Excitation of the donor fluorophore with a specific wavelength of light that does not excite the acceptor fluorophore can allow the donor to transfer its potential energy (called Forester resonance energy) to the acceptor molecule if it is within 10 nm of the donor. In some embodiments, when using BRET, the enzymatic activation of renilla luciferase (using a coelenterazine luciferin) is used as the energy source that is transferred to the acceptor, a fluorescent protein. In some embodiments, steric hindrance of large biomolecules could interfere with affinity interactions when using FRET or BRET.

In some embodiments, detection of affinity complexes does not require affinity labels (e.g. surface plasmon resonance spectroscopy (SPR), atomic force microscopy (AFM) or quartz crystal microbalance (QCM)). In some embodiments, the target is immobilized onto a surface, affinity reagents are added to the surface, and the affinity complex forms on the surface at the target. In some embodiments, binding of component molecules is observed and differentiated from binding of the entire complex. In embodiments using SPR, the addition of mass to the surface is detected by a change in the resonance angle, which is a function of the refractive index of the area immediately above the surface. In embodiments using AFM, a high resolution scanning probe technique physically measures minute structural changes of a surface using a cantilever. In embodiments using QCM, inorganic films are measured on surfaces. In some embodiments, a continuous-flow QCM system is used for the detection of biological interactions. In some embodiments, a microbalance is a piezoelectric quartz crystal that oscillates at a resonance frequency and this frequency changes with the addition of molecules to the surface of the crystal. In some embodiments, binding can be detected in real time as a solution containing the affinity reagents streams over the surface containing the target molecule.

### Therapeutics

In some embodiments, affinity binders are identified from very large DNA libraries using established panning methods. In some embodiments, affinity molecules are very stable, are easily modified or conjugated, and have low immunogenicity. In some embodiments, the compositions and method of embodiments of the invention are ideally suited for scaffold-based affinity therapeutics. All of the attributes afforded single domain affinity reagents are applicable to affinity complexes, but with improved binding affinity and specificity. In some embodiments, the small size of the components allows more rapid tissue penetration from the circulatory system and the lower affinities of the primary affinity molecules allows deeper penetration into, for example, a tumor (Thurber, et al. Adv Drug Deliv. Rev. 60, 1421-34). In some embodiments, the parameters of molecular size, binding affinity, clearance (both systematic and endocytic), and dosing effect tumor penetration. In some embodiments, small affinity molecules are highly soluble and allow high dosing of the therapeutic. In some embodiments, an optimum linker dose balances the thermodynamics of forming the affinity complex with penetration into a tissue. In some embodiments, systematic clearing of the small molecules is attenuated by including albumin or Fc binding domains to the molecules. In some embodiments, aspects of uniform therapeutic coverage are important for drug delivery, and for improved tumor or tissue imaging.

The versatility of embodiments of the invention as a therapeutic is demonstrated in the countless number of available affinity complexes. In some embodiments, at least two primary affinity molecules are used for complex formation. In some embodiments, as many primary affinity molecules as there are epitopes are used in a single reagent. In some embodiments, primary affinity molecules are interchangeable (e.g. Affibodies can be used with Monobodies or Microbodies or Nanobodies or DARPins, etc.). In some embodiments, linker molecules are interchangeable. In some embodiments, it is advantageous to rely on one or two affinity binders. In some embodiments, the linker molecule is two secondary affinity molecules tethered with a flexible polypeptide. In some embodiments, the linker molecule introduces a myriad of tags. In some embodiments, fluorescent dyes or proteins, radionucleotide or heavy metal chelates, and paramagnetic nanoparticles are used to image specific structures, tissues, and cells within the body. In some embodiments, drugs or toxins are used on the linkers to deliver their payloads directly to specific cells. In some embodiments, the therapeutic treatment ADEPT (antibody-directed enzyme prodrug therapy) is utilized with the present invention (e.g. an enzyme is added to the linker that catalyzes an inactive prodrug into its active form). In some embodiments, the Fc fragment of an IgG is fused to the linker molecule in order to activate natural cytotoxic cells.

In some embodiments, the present invention provides affinity molecules specific to cellular antigens that are expressed within a cell, termed intrabodies. In some embodiments, intrabody techniques have the capability to generate protein knockdown conditions similar to interference RNA (RNAi), phenotypic knockouts, and controlling specific biological processes in order to investigate genomic and proteomic functionality. In some embodiments, intracellular localization peptide sequences provide affinity molecules with the functionality to be directed to specific cell organelles (e.g. mitrochondria, nucleus, etc.) to improve their effect. In some embodiments, this technology requires that the gene coding sequences for the affinity molecules are introduced into the cells. In some embodiments, cell-permeable peptides (CPP) or protein transduction domains (PTD) connected to affinity molecules obviate the need to introduce genes encoding sequences for the affinity molecules into cells. In some embodiments, transducible affinity reagents, sometimes called transbodies, bind to the surface of a cell and are internalized by endocytosis. In some embodiments, affinity complexes, in which each component is tagged with a CPP or PTD are internalized, and allowed to form the affinity complex at the site of the target. In some embodiments, this approach significantly increases the effectiveness of this tool as a therapeutic, as well as a research tool.

### Diagnostics

Immunochemistry is a major segment of in vitro diagnostics (IVD) and is dominated by immune response derived antibodies (polyclonal and monoclonal). The cost of developing antibody mimics has for the most part precluded their use in this cost-conscious area, but could be advantageous where they can improve methodology, sensitivity, and practicality. Most IVD immunoassays are heterogeneous, requiring a surface (a plastic well, glass slide, bead) to which a reagent is attached that allows extensive washing to remove background contaminants and improve detection. Single-step homogeneous immunoassays have existed for over 20 years, but typically sacrifice sensitivity for convenience. Examples of older homogeneous immunoassays are fluorescence quenching or polarization (Abbott's TDS assay) and enzymatic steric hindrance assays (Miles Laboratories). FRET and BRET are more recent technologies to enter this field as well as a plethora of protein-fragment complementation assays (PCA). More recently, a highly sensitive one-step homogeneous immunoassay called nanoDLSA that measures the degree of nanoparticle aggregation by dynamic light scattering has been developed, but not commercialized (Liu et al. J. Am. Chem. Soc. 2008, 130, 2780-2782).

In some embodiments, the present invention finds utility in highly sensitive homogeneous immunochemistry assays. In some embodiments, affinity complexes for BRET and FRET have been described above and improve the versatility of these assays by labeling the secondary affinity molecules in a quinary affinity complex using coiled-coil or similar association mechanism. In some embodiments, PCA assays are suited for affinity complex dynamics, in which the affinity complex is a quinary system in which the complementary protein fragments have a specific affinity for each other. In some embodiments, enzymes that have been developed for this assay include β-galactosidase, β-lactamase, and renilla or firefly luciferase as well as green fluorescent protein (GFP) as a non-enzymatic example. In some embodiments, one fragment of the enzyme, or fluorescent protein, is expressed as a fusion or covalently linked to a secondary affinity molecule that binds to the primary affinity molecule and the other to another secondary affinity molecule. In some embodiments, the secondary affinity molecules of the two conjugates interact with different domains of the primary affinity molecules, otherwise the same fragment can be present on the same target (50%) resulting in a lack of signal.

### Systems and Kits

The present invention further provides systems and kits (e.g., commercial therapeutic, diagnostic, or research products, reaction mixtures, etc.) that contain one or more or all components sufficient, necessary, or useful to practice any of the methods described herein. These systems and kits may include buffers, detection/imaging components, positive/negative control reagents, instructions, software, hardware, packaging, or other desired components.

### EXPERIMENTAL

### Example 1

### Exemplary Use as a Therapeutic

A specific example of a therapeutic application of the invention involves a reagent that interacts with a tumor-specific cell surface protein. Trastuzumab, more commonly known as Herceptin (Genentech), is a humanized monoclonal antibody that recognizes human epidermal growth factor receptor-2 (HER2/neu or erbB-2) when it is over-expressed in aggressive tumors that occur in 20 to 25% of the estimated 200,000 breast cancer cases per year. HER2/neu is a large (p185) orphan receptor-based tyrosine kinase that forms heterodimers with other erbB receptors on the cell surface to effect cell differentiation and growth. A therapeutic product is created that contains at least two, and potentially more, Monobodies with binary code interfaces that interact with discontinuous epitopes of the HER2/neu protein, each with a KD of ˜10 nM. Each monobody has a 46 amino acid serum albumin binding domain (ABD) linked to its C-terminus that would extend serum half-life of the molecules. The therapeutic reagent contains a linker molecule consisting of a single chain, bimolecular Affibody with a 20 amino acid Gly/Ser linker that interacts with the EF loop of the monobody at a KD of ˜10 nM. The designed linker interacts with only 2 Monobodies at a time, but can form a complex with several different Monobodies, generating a “web” that blocks the receptor's dimerization and, therefore, signal transduction pathway. The linker molecule contains a high-contrast radionuclide imaging agent, such as 111indium chelated to benzyl-diethylenetriaminepentaacetic acid (DTPA), to track the affinity complex formation by positron emission tomography.

### Example 2

### Exemplary Use as a Diagnostic

A specific example of a diagnostic application of the invention involves a reagent that contains at least 2, and potentially more, Monobodies with binary code interfaces that interact with discontinuous epitopes of the protein interleukin-6 (IL-6, a modulator of inflammation), each with a KD of ˜10 nM. The reagent contains 2 more secondary Monobodies with binary code interfaces that specifically interact with the EF loop of the primary Monobodies (but not the secondary Monobodies) at a KD of ˜10 nM. The secondary Monobodies are linked via a Gly/Ser linker to a fragment of firefly luciferase, either N-luc or C-luc. The reagent contains components to reduce non-specific interactions (such as serum albumin), a buffer optimal for both binding interactions and luciferase activity, and the substrates luciferin and ATP. A sample of blood or serum is diluted into the reagent, the complexes are allowed to form on the IL-6, the proximity luciferase fragments allowed to complement/fold together, and complete enzymes generate bioluminescent light from the reaction with ATP and luciferin. The amount of sustained light output from this homogeneous assay is directly relative to the target protein concentration in the reaction. The volume of the reaction can be as large as 100 μl for use in a 96-well microtiter plate luminometer or less than a microliter for use in microfluidic devices. In addition, other targets can be assayed by simply changing the primary Monobodies in the reagent. This can also be used to assay membrane proteins on the surface of intact cells, such as GPCR or other receptors, and detect protein:protein interactions.

### REFERENCES

The following references are herein incorporated by reference in their entireties:
Handbook of Therapeutic Antibodies, 2007, ed. Stefan Dubel, Wiley-VCH
Nuttall & Walsh. Curr. Opin. Pharmacol. (2008) 8(5), 609-15.
Binz et al. Nat. Biotech. (2005) 23(10), 1257-68
Skerra. Curr. Opin. Biotechnol. (2007) 18(4), 295-304.
Hogbom et al. Proc. Nat. Acad. Sci. (2003) 100(6), 3191-6.
Skerra. Curr. Opin. Mol. Ther. (2007) 9(4), 336-44.
Silverman et al. Nat. Biotech. 23, 1556-61.
Koide et al. J. Mol. Biol. (1998) 284(4):1141-51.
Stumpp & Amstutz. Curr. Opin. Drug Discov. Devel. (2007) 10(2):153-9.
Roovers et al. Curr. Opin. Mol. Ther. (2007) 9(4), 327-35.
Nuttal, S D, et al., Proteins (1999) 36, 217-27.
Kolmar. FEBS J. (2008) 275(11), 2684-90.
Sergeeva et al. Adv. Drug Deliv. Rev. (2006) 58(15), 1622-1654.
Rothe et al. The FASEB J. (2006) 20, 1599-1610.
Thei et al. Curr. Pharm. Biotechnol. (2008) 9(6), 439-46.
Sidhu & Koide. Curr. Opin. Struct. Biol. (2007) 17(4), 481-7.
Gai & Wittrup. Curr. Opin. Struct. Biol. (2007) 17(4), 467-73.
Daugherty. Curr. Opin. Struct. Biol. (2007) 17(4), 474-80.
He & Khan, F. Expert Rev. Proteomics (2005) 2(3), 421-30.
Liu et al. Meth. Enzymol. (2000) 318, 268-93.
Suter et al. Curr. Opin. Biotechnol. (2008) 19(4), 316-23.
Ciruela. Curr. Opin. Biotechnol. (2008) 19(4), 338-43.
Bailon & Won. Expert Opin. Drug Deliv. (2009) 6(1), 1-16.
Veronese & Mero. BioDrugs (2008) 22(5), 315-29.
Porcheddu & Giacomelli. Curr. Med. Chem. (2005) 12(22), 2561-99.
Gullberg et al. Curr. Opin. Biotechnol. (2003) 14(1), 82-6.
Thøgersen & Holldack. Innovations in Pharmaceutical Technology: Drug Discovery (2006) vol. February, p. 27-31
Zhang et al. J. Mol. Biol. 335, 49-56.
Williamson. Nat. Chem. Biol. 4(8), 458-465.
Lamla & Erdmann. Protein Expr. Purif. (2004) 33(1), 39-47.
Yu. Adv. Drug Deliv. Rev. (2002) 54(8), 1113-29
Koide et al. Proc. Nat. Acad. Sci. (2007) 104(16), 6632-6637
Gilbreath. J. Mol. Biol. (2008) 381, 407-18.
Karlsson. J. Mol. Recognit. (2004) 17(3), 151-61.
Hinterdorfer & Dufrene. Nat. Meth. (2006) 3, 347-355.
Marx. Biomacromolecules (2003) 4(5), 1099-120.
Mishnick. Curr. Opin. Struct. Biol. (2001) 11(4), 472-7.
Liu et al. J. Am. Chem. Soc., (2008) 130 (9), 2780-2.
Stewart et al. Org. Biomol. Chem. (2008) 6(13), 2242-55.

We claim:

1. An affinity complex composition comprising providing (a) two or more primary affinity molecules, wherein said primary affinity molecules have affinity for one or more target molecules; and (b) one or more linker affinity molecules, wherein said linker affinity molecules have affinity for two or more of said primary affinity molecules; wherein said linker affinity molecules form a bridge when bound to two or more of said primary affinity molecules.

2. The affinity complex composition of claim 1, wherein said affinity complex composition preferentially assembles into a complex upon binding to a target molecule.

3. The affinity complex composition of claim 1, wherein said primary affinity molecules are selected from the list of natural antibodies, antibody fragments, single chain variable fragments, Nanobodies, Affibodies, Anticalins, DARPins, Monobodies, Avimers, and Microbodies.

4. The affinity complex composition of claim 1, wherein said primary affinity molecules are expressed from genes.

5. The affinity complex composition of claim 1, wherein said two or more primary affinity molecules comprise the same type of affinity molecule.

6. The affinity complex composition of claim 1, wherein said two or more primary affinity molecules comprise two or more types of affinity molecules.

7. The affinity complex composition of claim 1, wherein said two or more primary affinity molecules bind to different epitopes on said target molecule.

8. The affinity complex composition of claim 1, wherein said two or more primary affinity molecules bind to the same epitope on a target molecule.

9. The affinity complex composition of claim 8, wherein said target is a homomultimer and said epitopes are discontinuous.

10. The affinity complex composition of claim 1, wherein said linker affinity molecule comprises two or more secondary affinity domains that are linked.

11. The affinity complex composition of claim 10, wherein said secondary affinity domains bind said primary affinity molecules at an epitope.

12. The affinity complex composition of claim 11, wherein binding of said secondary affinity domains to said primary affinity molecules does not disrupt the function of said primary affinity molecules.

13. The affinity complex composition of claim 10, wherein said secondary affinity domains of said linker affinity molecule are covalently attached by a flexible linker.

14. The affinity complex composition of claim 13, wherein said flexible linker is selected from the list of polypeptide, a nucleic acid strand, polyethylene glycol, and peptide nucleic acid (PNA).

15. The affinity complex composition of claim 14, wherein said secondary affinity domains and said flexible linker comprise a single polypeptide.

16. The affinity complex composition of claim 14, wherein said secondary affinity domains and said flexible linker are attached through a non-covalent interaction.

17. The affinity complex composition of claim 1, further comprising one or more accessory molecules.

18. The affinity complex composition of claim 17, wherein said accessory molecules are selected from the list of fluorescent molecules, radioactive molecules, enzymes, inhibitors, drug molecules, and cell adhesion molecules.

19. The affinity complex composition of claim 17, wherein said accessory molecules are bound to said linker affinity molecule or to one or more of said primary affinity molecules.

20. The affinity complex composition of claim 17, wherein said accessory molecules comprise two or more fluorescent molecules linked to two or more affinity molecules.

21. The affinity complex composition of claim 20, wherein FRET can be detected between said fluorescent molecules upon formation of said affinity complex composition.

22. The affinity complex composition of claim 17, wherein said accessory molecules comprise a drug molecule, and wherein said drug molecule is incorporated into a cell upon interaction of said affinity complex composition with said target.

23. The affinity complex composition of claim 1, wherein said linker affinity molecule comprises an affinity complex comprising two or more secondary affinity molecules, wherein said secondary affinity molecules have affinity for each other.

24. The affinity complex composition of claim 23, wherein said secondary affinity molecules comprise coiled coil peptide sequences that interact in a specific manner.

25. The affinity complex composition of claim 23, wherein said secondary affinity molecules comprise complementary nucleic acid sequences.

26. The affinity complex composition of claim 23, wherein each said secondary affinity molecule has affinity for a tertiary affinity molecule.

27. The affinity complex composition of claim 26, wherein binding of said tertiary affinity molecule to said secondary affinity molecules does not interfere with binding of said secondary affinity molecules to said primary affinity molecules.

28. The affinity complex composition of claim 23, wherein said secondary affinity molecules interact with more than one domain of said primary affinity molecule.

29. The affinity complex composition of claim 1, wherein said affinity complex composition comprises a tyrosine/serine binary-code interface.

30. The affinity complex composition of claim 1, wherein said affinity complex composition comprises a tyrosine/serine/X amino acid tertiary-code interface.

31. The affinity complex composition of claim 1, wherein the interaction of a paratope of said primary affinity molecule to an epitope of said target molecule causes a change in the conformation of the primary affinity molecule to increase affinity of the linker interaction domain with the linker affinity molecule.

32. The affinity complex composition of claim 1, wherein standard cellular process causes a change in the conformation of said primary affinity molecule to increase the affinity of its linker interaction domain with said linker affinity molecule.

33. The affinity complex composition of claim 32, wherein said standard cellular process comprises: a protease, kinase, or phosphatase reaction; or a metal binding event.

34. The affinity complex composition of claim 1, wherein said affinity complex composition comprises three primary affinity molecules, wherein each of said primary affinity molecules bind to discontinuous epitopes of said target, and wherein said linker affinity molecules binds to said primary affinity molecules through secondary affinity molecules.
